(19) United States
(12) Patent Application Publication
(10) Pub. No.: US 2025/0032208 A1
(43) Pub. Date: Jan. 30, 2025

(54) MOBILE SYSTEM FOR BILATERAL ROBOTIC TOOL FEEDING

(71) Applicant: LEM Surgical AG, Bern (CH)

(72) Inventor: Yossi BAR, Muri bei Bern (CH)

(73) Assignee: LEM Surgical AG, Bern (CH)

(21) Appl. No.: 18/798,357

(22) Filed: Aug. 8, 2024

Related U.S. Application Data
(63) Continuation-in-part of application No. PCT/IB2022/058980, filed on Sep. 22, 2022.
(60) Provisional application No. 63/308,517, filed on Feb. 10, 2022.

(51) Int. Cl. A61B 50/13; A61B 34/30; A61B 90/50

(52) U.S. Cl. CPC A61B 50/13; A61B 34/30; A61B 90/50

(57) ABSTRACT

A tool feeding system includes a tool cart having a tool cart coordinate space and surgical tools removably carried by the tool cart at known locations within the tool cart coordinate space. The tool cart is physically attached to a surgical robotic system having one or more surgical robotic arms and a central control unit which controls movement of the robotic arms in a surgical robotic coordinate space. The central control unit registers the tool cart coordinate space with the surgical robotic coordinate space to create a common surgical robotic space, allowing the central control unit to kinematically control motion of the surgical robotic arms within the common surgical robotic space to retrieve and return multiple tools and perform surgical procedures with the tools.

This application is a continuation-in-part of PCT Application No. PCT/IB2022/058980, filed on Sep. 22, 2022, claiming the benefit of U.S. Provisional Application No. 63/308,517, filed on Feb. 10, 2022, the full disclosures of each of which is incorporated herein by reference in their entirety.

### BACKGROUND

### 1. Field

The disclosed technology relates generally to medical apparatus and methods and more particularly to surgical robot systems and apparatus and methods for providing surgical tool to surgical robotic system.

Robotic surgery and surgical robots are now in common use. A typical robotic surgical procedure requires deploying one or more surgical tools and end effectors on multiple surgical robotic arms using one or more robotically controlled cameras/sensors that may be located at varying distances and angulations from the surgical field. Accurate and precise placement of each of the tools and end effectors is necessary for the safe and successful completion of such robotic surgical procedures.

Robotic surgical procedures often require the use of multiple, different tool sets. For example, robotic placement of pedicle screws in a patient's spine may require the use of different screw types in each vertebra where each different pedicle screw may be configured for use with a unique tool set. The use of numerous different surgical tools and tool types necessitates multiple tool changes during a single surgical procedure, i.e., each tool or tool type needs to be connected to surgical robotic arm and then disconnected before loading a replacement tool. Presently, the needed tool changes are typically performed manually by the surgical team which can both extend the procedure time and increase the procedure cost.

For these reasons, it would be desirable to provided surgical robotic systems and methods where the loading and exchange of surgical tools on the surgical robotic arms can be at least partly automated, i.e., controlled and implemented by the surgical robotic controller with minimum control by the surgeon or surgical team. Such systems and methods should also be at least partially autonomous, e.g., being implemented under kinematic control with minimum or no reliance on optical or other sensor-based control protocols. Optical scanning of the tools and control of the surgical robotic arms is subject to limits on visibility, e.g., surgical personnel or a robotic arm can interfere with line-of-sight visibility of the tools, particularly when the tools are being held remotely from the surgical space. It would be further desirable if inventories of tool sets intended for specific procedures could be maintained together, allowing the inventories to be quickly exchanged at the outset and/or during an individual procedure. At least some of these objectives will be met by the technologies described and claimed herein.

### 2. Background Art

Background art includes patent publication nos. WO2019/096933; US2015/119637; US2018/168757; and WO2004/014244. Commonly owned publications and applications describing surgical robots and tools include PCT application nos. PCT/IB2022/052297 (published as WO2022/195460); PCT/IB2022/058986 (published as WO2023/067415); PCT/IB2022/058972 (published as WO2023/118984); PCT/IB2022/058982 (published as WO2023/118985); PCT/IB2022/058978 (published as WO2023/144602); PCT/IB2022/058980 (published as WO2023/152561); PCT/IB2023/055047 (published as WO2023/223215); PCT/IB2022/058988 (published as WO2023/237922); PCT/IB2023/055439; PCT/IB2023/055662; PCT/EP2024/052338; PCT/IB2023/055663; PCT/EP2024/052338; PCT/IB2023/056911; PCT/EP2024/052353; and US provisional application nos. 63/532,753, 63/568,102, 63/578,395; 63/606,001; 63/609,490; 63/615,076; 63/634161, the full disclosures of each of which are incorporated herein by reference in their entirety.

### SUMMARY

The disclosed technology provides variations and improvements to systems for performing robotically controlled and coordinated surgical procedures. In particular, the disclosed technology provides robotic systems which may comprise one or more robotic elements, such as surgical robotic arms, end effectors, tool holders, surgical instruments, cameras, imaging devices, tracking devices, or other devices useful for robotic surgery, and in particular for robotic spinal surgery. The disclosed robotic systems typically control placement and movement of the robotic elements using a single control unit where all of the robotic elements, including those integral to the surgical robot (such as the surgical robotic arms) as well as those that detachably attached to the surgical robotic arms (such as tools, tool holders, and end effectors) are controlled within a single (common) surgical robotic coordinate space, allowing all components of the surgical robotic system to be robotically coordinated from a single origin point. The surgical robot systems disclosed herein can employ any type of robotic coordinate system, including cartesian systems, polar coordinate systems, and the like. More particularly, the disclosed technologies provide a centrally coordinated and controlled surgical robotic system that is able to select and deploy tools from interchangeable adjacent carts with minimal disruption of the robotic surgical procedure.

Specifically, multiple robotic elements may be attached to, and controlled by, a single control unit and may be used in a coordinated fashion to deploy and/or relate to surgical instruments, trackers, cameras, and other surgical tools as part of a robotic surgical procedure. More particularly, in the context of robotic spinal surgery, multiple end effectors may be deployed on multiple robotic arms and controlled by a single control unit and may be used in a centrally coordinated fashion to perform a robotic surgical procedure, with the relative movements of each robotic element being coordinated by the central control unit. Most particularly, in the context of a robotic spinal surgery procedure requiring high accuracy, repetitive tasks and multiple tool sets, the disclosed technology relates to a robotic system wherein tool carts adjacent to the central robotic chassis may be interchanged during the surgery and the robotic system may select and feed itself tools and deploy them efficiently and accurately during the surgical procedure.

In a first aspect, the disclosed technology provides a tool feeding system intended to be used with a robotic surgical system which includes one or more surgical robotic arms mounted on one or more chasses and which has a central control unit configured to control movement of the robotic arms in a surgical robotic coordinate space. The tool feeding system comprises at least one tool cart having a tool cart coordinate space and is configured to be physically attached to and detached from the surgical robotic system. A plurality of surgical tools are removably carried by the least one tool cart at locations within the tool cart coordinate space, and the central control unit is configured to register the tool cart coordinate space with the surgical robotic coordinate space to provide a common surgical robotic coordinate space and to kinematically control motion of the one or more surgical robotic arms within the common surgical robotic coordinate space.

“Kinematic” positioning and tracking of the surgical robotic arms and surgical tools within the common surgical robotic coordinate space means that the central control unit positions and tracks the distal ends of the robotic surgical arms primarily or solely based upon the dimensions, geometries, and of the robotic arms without the need to optically or otherwise track movement of the distal end. For example, in the typical case of articulated robotic arms, the controller can calculate and track the position of the distal end of each arm based upon the dimensions of each component or link of the arm and the direction and degree of bending between adjacent components. By further knowing the dimensions of the tools and any related tool holders, end effectors, and other components attached to the surgical robotic arms, the positions of all such components in the common surgical robotic coordinate space can be calculated and tracked. Such kinematic tracking of the robotic arms is well known in the art of surgical and other robots and needs no further description.

While the central control unit can kinematically position and track the surgical robotic arms for the purpose of locating, acquiring and exchanging surgical tools on a tool cart as described above, the positions of the robotic surgical arms as well as the patient anatomy, surgical personnel, surgical tools and other objects attached to the surgical arms or not, may also be tracked by a camera or the sensors. As the position of the camera/sensor itself may typically be kinematically tracked, the positions of the objects observed by the camera/sensors can be used to confirm and/or in addition to the direct kinematic tracking.

In some instances, the tool feeding system may be configured for use with a robotic surgical system comprising one or more surgical robotic arms mounted on a single chassis.

In some instances, the tool feeding system may be configured for use with a robotic surgical system comprising one or more surgical robotic arms mounted on two or more chasses which are configured to be physically attached to each other to form a single surgical robotic coordinate space.

In some instances, the tool feeding system may be configured for use with a robotic surgical system having the central control unit mounted on the one or more chasses.

In some instances, the tool feeding system may be configured for use with a robotic surgical system having the central control unit located remotely from the one or more chasses.

In some instances, the central control unit may be configured to perform at least the following operations: (1) retrieve surgical tools from the at least one tool cart, (2) manipulate the tools in the surgical robotic coordinate space, and (3) return said surgical tools to tool. For example, the central control unit may be configured to perform at least some of said operations via a used input interface that allows a user to manipulate the surgical robotic arms. Alternatively, the central control unit may be configured to perform at least some of said operations automatically in response to a control program, typically being programmed with the locations of individual tools and/or tool types within the common surgical robotic space to allow the central control unit to retrieve and/or return specific tools to the at least one tool cart based upon said locations.

In some instances, the control program may be programmed with the locations of individual tools and/or tool types within the common surgical robotic space to allow the central control unit to retrieve and/or return specific tools to the at least one tool cart based upon said locations.

In some instances, the tool feeding system may be configured for use with a robotic surgical system comprising one or more cameras and wherein the central control unit is configured to optically track the surgical tools in addition kinematically tracking said surgical tools. In such instances, registering the tool cart coordinate space with the surgical robotic coordinate space to provide a common surgical robotic space may comprise optically registering marker on the robotic chassis and the tool cart in addition to kinematic registration.

In some instances, the central control unit may be further configured to control the inventory of surgical tools carried by the at least one tool cart.

In a second aspect, the disclosed technology provides a method for feeding tools to a robotic surgical system comprising one or more surgical robotic arms mounted on one or more chasses and having a central control unit which controls movement of the robotic arms in a surgical robotic coordinate space. The method may comprise attaching at least one tool cart to the chassis of the robotic surgical system and registering a tool cart coordinate space of the tool cart with the surgical robotic coordinate space of the robotic surgical system to form a common surgical robotic space. The tool cart can removably carry a plurality of surgical tools at known locations within the tool cart coordinate space, and the surgical robotic arms may be kinematically manipulated to locate and retrieve individual tools from the tool cart based upon the tool's known retrieval location in the common surgical robotic space. The one or more of the surgical robotic arms may then be further manipulated to perform a surgical operation on a patient with the retrieved surgical tools. The tools may be interchanged as needed during the procedure by returning the tools to the cart (or to a temporary holding location) and retrieving other tools with the freed robotic arm. At the end of the procedure, the one or more surgical robotic arms may be kinematically manipulated to return individual tools to the tool cart based upon the tool's return location in the common surgical robotic space.

In some instances, manipulating the one or more surgical robotic arms to perform the surgical operation on a patient with the retrieved surgical tools may comprise at least in part kinematically tracking the locations of said retrieved surgical tools and said surgical robotic arms.

In some instances, manipulating the one or more surgical robotic arms to perform the surgical operation on a patient with the retrieved surgical tools may comprise at least in part optically tracking the locations of said retrieved surgical tools and said surgical robotic arms.

In some instances, attaching the at least one tool cart to the chassis of the robotic surgical system may comprise detachably securing a chassis of the tool cart to the chassis of the robotic surgical system with a known arrangement that allows the central control unit to register the tool cart coordinate space with the surgical robotic coordinate space to form the common surgical robotic space.

In some instances, registering the tool cart coordinate space of the tool cart with the surgical robotic coordinate space of the robotic surgical system to form a common surgical robotic space may comprise at least in part optically registering markers on the tool cart with markers on the robotic surgical system.

In some instances, the robotic surgical system may comprise at least one mobile cart where the mobile cart may be positioned adjacent to and/or under a surgical table prior to attaching at least one tool cart to the chassis.

In some instances, the robotic surgical system may be mounted on a surgical bed.

In some instances, the retrieval and return locations are the same. In other instances, the retrieval and return locations may be different.

Provided herein is a mobile robotically controlled surgical system. In some embodiments, the disclosed system is a centrally coordinated and synchronized robotic system for spinal robotic surgery procedures, optionally for bilateral approach in spinal robotic surgery procedures. The system may comprise multiple robotic arms that each can hold, place and/or manipulate at least one end effector, camera or navigation element for use in a spinal surgery procedure. The end effectors may include any surgical tools useful for performing spinal surgical procedures and are interchangeable. The cameras and navigation elements may be for another layer of accuracy and confidence providing guidance for the movement of the robotic arms and deployment of the end effectors and tools.

The disclosed technology comprises multiple robotic arms which access and visualize the surgical field in an automatic and safe way because they are robotically synchronized. In one embodiment, there may be two robotic arms, one of which place, guide and/or hold end effectors and/or tools and one holding a navigation and imaging camera. In another embodiment, there are three arms in which two are using different surgical tools and different end effectors. In such an embodiment, the arms holding the tools may, after the tools have been placed, bring and manipulate other end effectors or tools in the surgical field. In such an embodiment, the first arm may optionally position and then control the use of, for example, a drilling tool. The second arm may optionally position and then control the placement of an element such as a screwdriver. A third arm may optionally hold a camera that provides an image of the process from an optimal distance and angulation. The camera is able to operate from optimal distance and angulation because it is sized appropriately and its deployment on an appropriately sized and positioned robotic arm. Optionally, the robotic arms may also hold additional imaging or navigation cameras to provide redundancy and diversity of information. Also optionally, the robotic arms and/or the tools or end effectors may have active or passive markers placed on them that may assist the robotic system in positioning the robotic arms, the tools and/or the end effectors.

In one embodiment, the already robotically synchronized movement of the robotic arms is enhanced by the interaction of the navigation cameras with active or passive markers that are placed during or at the beginning of the procedure on portions of the patient's anatomy. The movement of the robotic arms is synchronized by a central control unit from a single base that knows where the arms are based upon. The additional navigation information provided by the various markers and the one or more cameras can improve that accuracy in some cases or add another layer of protection and verification.

In some embodiments of the disclosed technology, carts with robotic tool sets may be brought into the surgical field and optionally attached and detached from the central chassis of the robotic surgical system using a robust, accurate and repeatable mechanical and electrical connection. Once rigidly mechanically attached to the central chassis, the tool carts may be essentially robotically synchronized with the robotic surgical system. In this regard, the multiple robotic arms are able to accurately select and deploy tools from the tool carts into the surgical field in a centrally coordinated manner. In alternative embodiments, tool carts containing tool sets that are no longer needed during the surgical procedure may be detached from the central chassis and alternative tool carts with alternative tool sets may be brought into the surgical field and may be rigidly mechanically attached to the central robotic chassis. Again, once attached, the new tool carts are robotically synchronized with the robotic surgical system and the multiple robotic arms are able to select and deploy the tools in a centrally coordinated manner. In these representative embodiments of the disclosed technology, multiple mobile, portable, variable and versatile tool sets can be brought into the surgical field and subsequently removed without impacting operating room floor space or surgical workflow too dramatically.

In an alternative embodiment of the disclosed technology, the central robotic single chassis system can be equipped with a barcode or RFID scanner that is configured to be able to read appropriate RFID codes on surgical tools or tool carts. This allows the robotic system to keep an inventory of available tools and implants that have been used in the surgical procedure and also to prevent mistakes of using wrong tools. In this embodiment, the centrally coordinated robotic system is able to check that the desired tools were selected and used and, thus, provide a safety and accuracy check with respect to the surgical procedure. In the example of complex spinal surgical procedures involving repetitive tasks with multiple different tool sets, this functionality can only be said to enhance accuracy, surgeon performance and patient safety. This functionality is enabled by the scanner device and tool carts being optionally connected to the central robotic chassis, which in turn can then centrally coordinate the progress of the surgical procedure.

The disclosed embodiments take advantage of multiple feedback loops to ensure precision and safety in the performance of a bilateral robotic spinal surgical procedure. The movement of the robotic arms is robotically synchronized to the greatest possible level of precision because the relatively small robotic arms are all co-mounted on a single rigid chassis that has a central control unit.

The robotic arms bases are also mounted on the central chassis relatively far from each other, for example at least one meter apart-thus providing for greater reachability, maneuverability and moments application. This advantage is significant since being able to have several robotic arms which are not very big e.g., up to one meter reach but their bases are relatively far from each other enables high reachability and area coverage of f the surgical room with relatively small arms while being highly accurate and without notable interference for the surgical staff. Robotic navigation is provided by one or more cameras/sensors that are deployed by one or more robotic arms that are also co-mounted on the same single chassis and are also controlled by the same central control unit. All of these needs and elements benefit tremendously from the central coordination and synchronized control of the disclosed mobile single-cart, multi-arm, non-teleoperated robotic system. Based on the placement of appropriately sized markers and the placement of navigation cameras at an appropriate distance and orientation to the target anatomy and the markers, movement of the robotic arms carrying end effectors and cameras can be further coordinated to provide for a safe and precise robotic spinal surgical procedure. Rigid mechanical attachment of interchangeable tool carts to the central chassis, along with the use of scanning tool inventory elements allows for an unprecedented level of accuracy and diversity in the performance of repetitive tasks in spinal robotic surgery.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an end view of a robotic spinal surgery system with multiple tool cart elements being available and interchangeably connected according to some embodiments of the disclosed technology.

FIG. 2 is an alternative overhead view of a robotic spinal surgery system with interchangeable tool carts according to some embodiments of the disclosed technology.

### DETAILED DESCRIPTION OF THE TECHNOLOGY

With reference now to the figures and several representative embodiments of the disclosed technology, the following detailed description is provided.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

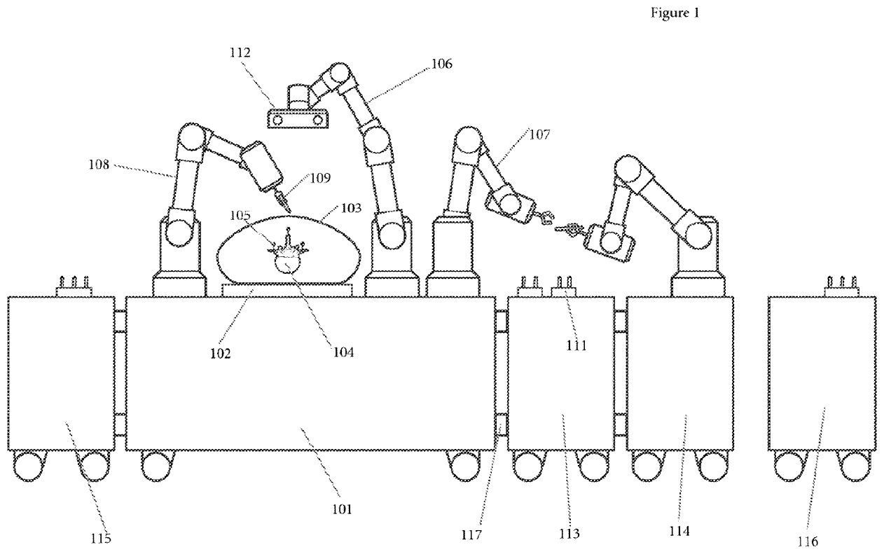

In a working example of the disclosed technology exemplified by FIG. 1, a mobile, bilateral robotic spinal surgery system is shown. The system comprises a mobile central chassis 101 that can be deployed under a surgical table 102. In a representative application, the body of a patient 103 is placed in on the surgical table. Shown in FIG. 1 is a vertebra 104 of the patient 103, wherein a surgical marker 105 may optionally be placed on the vertebra 104 of the patient 103. The optional surgical marker 105 may be used in conjunction with a navigation camera 112 to register the position of the patient 103 so that it will be known by a central control unit found in the central chassis 101 of the robotic surgical system. Another and alternative method to register the robotic arms to the patient anatomy does not require the navigation marker but another radiopaque marker placed on the robotic arm and being scanned together with the bony anatomy.

The robotic surgical system may further comprise three robotic arms 106, 107 and 108 that are all deployed on the central chassis 101. One of skill in the art will realize that more or less robotic arms can be deployed on the central chassis 101 (e.g., two arms from both sides both holding cameras bi-laterally) but that three arms may be suitable for many robotic spinal surgical procedures since two arms may hold robotic tools and one may hold a navigation camera. This three-arm configuration may be especially useful to those of skill in the art as it may optionally enable a bilateral approach to robotic spinal surgery. This bilateral approach may be enabled by the present robotic surgical system that positions a central chassis 101 under the surgical table allowing for relatively small and maneuverable robotic arms to approach the patient from either side of the table, all with a small operating room footprint.

FIG. 1 also shows several tool carts 113, 114, 115 and 116 as being aspects of an embodiment of the disclosed technology. The tool carts 113, 114, 115 and 116 may optionally be mechanically rigidly connected to each other and to the central chassis 101 by connection elements 117. The connection elements 117 may optionally include electronic communication means so that, among other things, the central control unit found in the central chassis 101 can identify the tool carts that are connected 113, 114, and 115, know their position, and guide the robotic arms 106, 107, 108 to select tools from the tool carts.

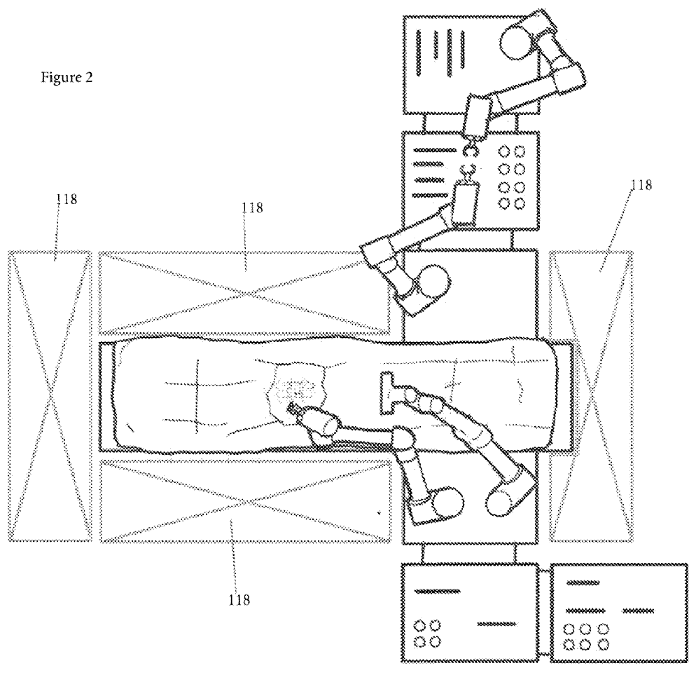

In the representative embodiment of FIG. 1, robotic arm 106 is holding a navigation camera 112, robotic arm 107 is positioned to select a tool from a tool cart 113, and robotic arm 108 is holding a tool 109. A representative alternative tool set 111 is positioned on tool cart 113. In addition, tool carts according to embodiments of the disclosed technology may comprise an additional robotic arm 110 that may be used for the positioning, exchange and deployment of surgical tools. FIG. 2 provides a top view of a similar embodiment to that found in FIG. 1. FIG. 2 provides one of skill in the art with a more comprehensive view of a representative layout of the disclosed system in an operating room. Specifically, FIG. 2 shows areas of operating room floor 118 that are unoccupied by the described system. The areas of unoccupied operating room floor 118 are thus available for medical staff and other surgical equipment, such as intraoperative imaging equipment. Moreover, since this is a mobile system in its essence, all carts can be quickly deployed in any surgical room and later quickly taken out and clearing the room for other types of surgery. FIG. 2 thus demonstrates that the current disclosed system provides significant advantages in terms of minimal footprint and minimal disruption of surgical workflow.

The disclosed system demonstrated in FIGS. 1 and 2 allows for the performance of novel and versatile robotic spinal surgical procedures. Solely by way of example, robotic spinal surgery often requires the placement of multiple pedicle screws or other hardware elements in multiple vertebrae of a patient. These are complex, repetitive tasks that can challenge even the best spine surgeon. Repetition and fatigue are factors in the performance of these types of procedures, as are the natural limits of human precision and accuracy. Surgical tasks such as these are further complicated by the fact that different areas of patient anatomy may require different surgical tools and different surgical approaches, thus necessitating the use of different tool sets. As the surgeon progresses through the patient's spinal column and places pedicle screws, they may wish to use different hardware types and may also wish to take different surgical approaches, thus necessitating the use of different tool sets. This type of surgical task may be greatly facilitated by a robotic system that can easily incorporate the use of different tool sets and also automates tool, thus providing accuracy beyond the limits of human performance.

One of skill in the art reading the present disclosure and viewing FIGS. 1 and 2 will understand that the surgical team may sequentially bring tool carts 113, 114, 115 and 116 into the surgical field, each bearing different tool sets. The surgical robotic system may then deploy the tool sets in a centrally coordinated manner that increases accuracy. One of skill in the art will realize that several variations on the disclosed embodiments are possible while staying within the bounds of the disclosed technology. Solely by way of example, different variations in the number of navigation cameras, robotic arms, markers and end effectors can be used without departing from the disclosed technology. As another example, markers of varying sizes can be used. As yet another example, numerous variations of surgical tools and surgical approaches to unilateral or bilateral spinal surgical can be employed without departing from the disclosed technology described herein. The embodiments provided are representative in nature.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1.-13. (canceled)

14. A method for feeding tools to a robotic surgical system comprising one or more surgical robotic arms mounted on one or more chassis and having a central control unit which controls movement of the one or more surgical robotic arms in a surgical robotic coordinate space, said method comprising:
attaching at least one tool cart to the chassis of the robotic surgical system;
registering a tool cart coordinate space of the at least one tool cart with the surgical robotic coordinate space of the robotic surgical system to form a common surgical robotic space, wherein the at least one tool cart removably carries a plurality of surgical tools at known locations within the tool cart coordinate space;
kinematically manipulating one or more of the one or more surgical robotic arms to locate and retrieve individual tools from the at least one tool cart based upon the individual tool's known retrieval location in the common surgical robotic space;
manipulating one or more of the one or more surgical robotic arms to perform a surgical operation on a patient with the retrieved surgical tools; and
kinematically manipulating the one or more surgical robotic arms to return individual tools to the at least one tool cart based upon the individual tool's return location in the common surgical robotic space.

15. The tool feeding method of claim 14, wherein manipulating the one or more surgical robotic arms to perform the surgical operation on a patient with the retrieved surgical tools comprises at least in part kinematically tracking the locations of said retrieved surgical tools and said one or more surgical robotic arms.

16. The tool feeding method of claim 14, wherein manipulating the one or more surgical robotic arms to perform the surgical operation on a patient with the retrieved surgical tools comprises at least in part optically tracking the locations of said retrieved surgical tools and said one or more surgical robotic arms.

17. The tool feeding method of claim 14, wherein attaching the at least one tool cart to the chassis of the robotic surgical system comprises detachably securing a chassis of the at least one tool cart to the chassis of the robotic surgical system with a known arrangement that allows the central control unit to register the tool cart coordinate space with the surgical robotic coordinate space to form the common surgical robotic space.

18. The tool feeding method of claim 14, wherein registering the tool cart coordinate space of the tool cart with the surgical robotic coordinate space of the robotic surgical system to form a common surgical robotic space comprises at least in part optically registering markers on the at least one tool cart with markers on the robotic surgical system.

19. The tool feeding method of claim 14, wherein the robotic surgical system comprises at least one mobile cart and wherein said at least one mobile cart is positioned adjacent to and/or under a surgical table prior to attaching at least one tool cart to the chassis.

20. The tool feeding method of claim 14, wherein the robotic surgical system is mounted on a surgical bed.

21. The tool feeding method of claim 14, wherein the retrieval and return locations are the same.

22. The tool feeding method of claim 14, wherein the retrieval and return locations are different.

23. A robotic tool feeding system comprising:
a centrally coordinated robotic system comprising at least two robotic arms mounted on a single rigid chassis containing a central control unit; and at least one tool cart physically connected to the robotic system;
wherein the physical connection of the at least one tool cart to the robotic system allows the central control unit to guide at least one of the at least two robotic arms to select a tool from the at least one tool cart and move it into a surgical field.

24. The robotic tool feeding system of claim 23, comprising at least three robotic arms mounted on the single rigid chassis.

25. The robotic tool feeding system of claim 24, wherein at least two of the at least three robotic arms hold end effectors and wherein at least one of the at least three robotic arms holds a navigation camera.

26. The robotic tool feeding system of claim 23, wherein the robotic system is mobile.

27. The robotic tool feeding system of claim 26, wherein the mobile robotic system can be selectively moved under a surgical table and removed from under the surgical table before, during, or after a surgical procedure.

28. The robotic tool feeding system of claim 23, further comprising at least two tool carts physically connected to the robotic system.

29. The robotic tool feeding system of claim 25, further comprising at least one surgical marker placed on patient anatomy of interest during a surgical procedure and wherein the locations of the at least two robotic arms are registered with the patient anatomy at least in part based on information provided to the central control unit by the navigation camera imaging the surgical marker.

30. The robotic tool feeding system of claim 23, wherein the central control unit guides the robotic system to select multiple tools from the at least one tool cart and to carry out multiple surgical steps on the patient.

31. The robotic tool feeding system of claim 23, further comprising a means for controlling inventory of tools on the at least one tool cart.

32. The robotic tool feeding system of claim 31, wherein the means for controlling inventory is RFID.

33. The robotic tool feeding system of claim 23, wherein one or more of the at least two robotic arms are configured to move one or more tools from one tool cart to another tool cart.

34. The robotic tool feeding system of claim 23, further comprising navigation markers placed on one or more tools located on the at least one tool cart.

35. The robotic tool feeding system of claim 23, further comprising navigation markers placed on one or more of the at least two robotic arms.
